(19) United States
(12) Patent Application Publication
(10) Pub. No.: US 2010/0144679 A1
(43) Pub. Date: Jun. 10, 2010

(54) MEDICATION KITS AND FORMULATIONS FOR PREVENTING, TREATING OR REDUCING SECONDARY FRACTURES AFTER PREVIOUS FRACTURE

(75) Inventor: Kenneth W. Lyles, Durham, NC (US)

Correspondence Address: MOORE & VAN ALLEN PLLC, P.O. BOX 13706, Research Triangle Park, NC, 27709, US

(73) Assignee: DUKE UNIVERSITY, RTP, NC (US)

(21) Appl. No.: 12/532,285

(22) PCT Filed: Mar. 21, 2008
(86) PCT No.: PCT/US08/57778

§ 371 Date: Feb. 15, 2010

Related U.S. Application Data
(60) Provisional application No. 60/896,058, filed on Mar. 21, 2007.

(51) Int. Cl. A61K 31/675

(52) U.S. Cl. 514/94

(57) ABSTRACT

The present invention provides a multi-component kit comprising a therapeutically effective amount of a bisphosphonate or analogue thereof in combination with an effective amount of Vitamin D, a metabolite thereof, a precursor thereof or an analogue thereof, wherein the kit provides instructions for daily dosing and correct chronological order of administration of components to provide a dosing regime for reducing secondary osteoporotic skeletal fractures in subjects at risk for vitamin D deficiency.

### CROSS REFERENCE TO RELATED APPLICATION

The present invention claims priority to U.S. Provisional Application No. 60/896,058 filed on Mar. 21, 2007, the contents of which are incorporated by reference herein for all purposes.

### BACKGROUND OF THE INVENTION

1. Field of the Invention

This invention relates to pharmaceutical formulations and kits comprising zoledronic acid or salts or hydrates thereof in combination with and Vitamin D and/or Calcium.

2. Description of the Related Art

Bisphosphonates are analogues of pyrophosphate and exhibit marked effects on bone metabolism. The bisphosphonates' characteristic phosphorus-carbon-phosphorus bond (P—C—P) renders the class resistant to hydrolysis by phosphatases and enables these molecules to bind tightly to calcified bone matrix. They are very effective inhibitors of osteoclastic bone resorption and have been used clinically in Paget's disease of bone, osteoporosis, hypercalcemia of malignancy and bone metastases. Zoledronic acid, i.e. 1-hydroxy-2-(imidazol-1-yl)ethane-1,1-d-iphosphonic acid, is a nitrogen containing bisphosphonate (third generation). In a variety of assays of bone containing metabolism, zoledronic acid has demonstrated inhibition of bone resorption in vitro at concentrations of 0.3-30 nM, and in vivo at doses of 0.3-30 ug/kg without exerting any untoward effects on either bone formation or mineralization.

Hip fractures are the most devastating of the osteoporotic fractures. Patients with hip fractures lose bone mass and muscle mass in the year following the fracture. There is a very high risk of subsequent fractures after hip fracture in both men and women. These secondary fractures significantly affect the quality of life of patients already struggling to recover from their initial hip fracture. Men and women hip fracture patients have much to gain from the development of an effective secondary fracture prevention intervention.

Although bisphosphonates have been found to be a useful treatment in bone disorders there is concern that many of the patients who receive this drug are deficient in vitamin D. Notably, unrecognized, untreated vitamin D deficiency can cause hypocalcemia, seizures and sometimes lead to death.

Thus, there is a need to develop a system wherein a bisphosphonate, such as Zoledronic acid, is administered while ensuring the patient is not further compromised by hypocalcemia.

### SUMMARY OF THE INVENTION

Accordingly the present invention provides a kit for reducing secondary osteoporotic skeletal fractures in subjects at risk for vitamin D deficiency by providing a therapeutically effective amount of a bisphosphonate or analogue thereof in combination with an effective amount of Vitamin D, a metabolite thereof, a precursor thereof or an analogue thereof. Preferably, the vitamin D is in the form of cholecalciferol, calcidiol, ergocalciferol, dihydrotachysterol, doxercalciferol, paricalcitol or calcitriol.

In one aspect, the present invention relates to a multi-component kit for a patient at risk for secondary osteoporotic skeletal fractures and vitamin D deficiency, the kit comprising (a) Vitamin D or a functional analogue thereof; (b) zoledronic acid or a functional analogue thereof; and optionally a calcium containing component.

In another aspect, the present invention relates to a method for protecting a patient at risk for secondary osteoporotic skeletal fractures and vitamin D deficiency comprising the steps of:
a) providing a container sized for inclusion of a sufficient amount of Vitamin D for a dosing regime comprising multiple dosages and a single dosage of a bisphosphonate; and
b) providing instructions for the dosing regime.

Preferably the container is configured to be permissive for the removal of a single dosage of Vitamin D on a daily basis and removal of same is evidenced by an empty dosage spot in the container. Still further, the container is preferably configured to include a tablet, transdermal patch, syringe or vial of a single dosage of zoledronic acid or analogue thereof.

In yet another aspect, the present invention relates to a composition kit comprising at least two portions wherein multiple doses of Vitamin D or an analogue thereof are packaged in a first portion and at least one dose of zoledronic acid is packaged in a second portion. Optionally, a third portion may include a calcium containing compound.

In a still further aspect the present invention relates to a kit, having a first and a second product, wherein the second product is administered to a subject after sequentially administering the first product and wherein the first product comprises a form of Vitamin D and the second product is zoledronic acid, a salt or hydrate thereof. Preferably, the kit provides sufficient doses for administering the first product for 10 to 31 days and a single dose of the second product after the administration of the first product.

The unit dose of Vitamin D will be determined by the specific form, the number of day of administration, age and condition of patient, and level of Vitamin D deficiency. The level of Vitamin D deficiency can be easy determined by a simple blood test that determines the level of Calcidiol (25-hydroxyvitamin D). For example, cholecalciferol may in a unit tablet dose of from about 400 to 5000 IU or in intramuscular form from about 50,000 units/cc to 100,000 units/cc; egocalciferol in unit capsule dose of from about 400 to 50,000 IU; oral calcitriol in a dose from about 0.10 to about 1 mcg which can be administered at least once a day or in multiple administrations; calcidiol or doxercalciferol, both of which are vitamin D analogues may be administered in dose units of from about 300 to 2000 IU.

Use of the kits of the present invention is particularly applicable to the patients who have had a hip fracture repair within the past 1-7 days, 60 days, past 90 days, 142 days, the past 545 days, past 742 days, and preferably wherein the hip fracture repair was within the past 90 days.

Another aspect of the invention relates to a method of treating bone disease and Vitamin D deficiency, comprising:
a) opening a sealed disposable package, wherein the package comprises multiple single-unit doses of Vitamin D and optionally calcium;
b) administering a single-unit dose, wherein steps a) to b) are repeated daily for a period of 10 to 31 days; and
c) opening a vial contained within the sealed disposable package, wherein the vial comprises a single-unit dose of zoledronic acid or analogue thereof.

In yet another aspect, the present invention relates to a formulation comprising an admixture of a form of vitamin D and zoledronic acid in therapeutically amounts, wherein the formulation is in a solid, powder, liquid or gel form.

Other aspects and advantages of the invention will be more fully apparent from the ensuing disclosure and appended claims

### DETAILED DESCRIPTION OF THE INVENTION

As defined herein, a “bisphosphonate” includes any compound which is an analog of endogenous pyrophosphate whereby the central oxygen is replaced by carbon. Bisphosphonates include aminobisphosphonates. Bisphosphonates include, but are not limited to the compounds zoledronic acid, risedronate, alendronate, cimadronate, clodronate, tiludronate, etidronate, ibandronate, piridronate or pamidronate.

Examples of pharmaceutically acceptable salts of the compounds include salts derived from an appropriate base, such as an alkali metal (for example, sodium, potassium), an alkaline earth metal (for example, calcium, magnesium), ammonium and NR′₄⁺ (wherein R′ is C₁-C₄ alkyl). Pharmaceutically acceptable salts of an amino group include salts of: organic carboxylic acids such as acetic, lactic, tartaric, malic, lactobionic, fumaric, and succinic acids; organic sulfonic acids such as methanesulfonic, ethanesulfonic, isethionic, benzenesulfonic and p-toluenesulfonic acids; and inorganic acids such as hydrochloric, hydrobromic, sulfuric, phosphoric and sulfamic acids. Pharmaceutically acceptable salts of a compound having a hydroxyl group consist of the anion of said compound in combination with a suitable cation such as Na⁺, NH₄⁺, or NR′₄⁺ (wherein R′ is for example a C₁₋₄ alkyl group).

As defined herein, the word “treatment” refers to inhibiting bone resorption or demineralization and/or the reduction or elimination of secondary fractures.

As defined herein, “therapeutic” means a treatment administered to a subject who exhibits signs of pathology for the purpose of diminishing or eliminating those signs.

The term “therapeutically effective amount,” as used herein means an amount of a bisphosphonate compound and/or a form of Vitamin D that is sufficient to provide a beneficial effect to the subject to which the compound is administered. A beneficial effect means reduction in secondary bone fractures after an initial bone fracture while reducing Vitamin D deficiency.

The term “a form of Vitamin D,” as used herein mean any from of Vitamin D and functionally active analogue including Vitamin D2 (ergocalciferol or calciferol) and Vitamin D3 (cholecalciferol); hormones including calcidiol, dihydrotachysterol and calcitriol; Vitamin D analogues or metabolites including doxercalciferol and paricalcitol.

The term “functionally active analog,” means compounds derived from a particular parent compound by straightforward substitutions that do not result in a substantial (i.e. more than 100×) loss in the biological activity of the parent compound, where such substitutions are modifications well-known to those skilled in the art, e.g., esterification, replacement of hydrogen by halogen, replacement of alkoxy by alkyl, replacement of alkyl by alkoxy, etc.

Zoledronic acid, as used herein, is intended to include the free acid itself, i.e., 1-hydroxy-2-(imidazol-1-yeethane-1,1-diphosphonic acid, as well as any pharmaceutically acceptable salts and hydrates thereof and solvates thereof forming from other solvents used for its crystallization. 1-hydroxy-2-(imidazol-1-yeethane-1,1-diphosphonic acid and its pharmacologically acceptable salts, hydrates and solvates are well-known from the literature. They can be prepared by procedures known in the art, such as described, e.g., in U.S. Pat. No. 4,939,130. See also U.S. Pat. Nos. 4,777,163 and 4,687,767. The contents of the latter three patents are hereby incorporated by reference in their entirety.

Especially preferred pharmaceutically acceptable salts are those where one, two, three or four, in particular one or two, of the acidic hydrogens of the zoledronic acid are replaced by a pharmaceutically acceptable cation, in particular sodium, potassium or ammonium, in the first instance sodium.

A more preferred group of pharmaceutically acceptable salts is characterized by having one acidic hydrogen and one pharmaceutically acceptable cation, especially sodium, in each of the phosphonic acid groups.

Zoledronic acid is preferably used in the form of pharmaceutical compositions that contain a therapeutically effective amount of zoledronic acid active ingredient optionally together with or in admixture with inorganic or organic, solid or liquid, pharmaceutically acceptable carriers which are suitable for administration.

The pharmaceutical compositions may be, for example, compositions for enteral, such as oral, rectal, aerosol inhalation or nasal administration, compositions for parenteral, such as intravenous or subcutaneous administration, or compositions for transdermal administration, e.g., passive or iontophoretic.

Preferably, the pharmaceutical compositions are adapted to oral or parenteral administration. Intravenous and oral, first and foremost intravenous, administration is considered to be of particular importance. Preferably the zoledronic acid active ingredient is in the form of a parenteral, most preferably an intravenous form.

The particular mode of administration and the dosage may be selected by the attending physician taking into account the particulars of the patient, especially age, weight, life style, activity level, hormonal status, e.g., post-menopausal, and bone mineral density as appropriate. Most preferably, however, the zoledronic acid is administered intravenously.

The dosage of the zoledronic acid may depend on various factors, mode of administration, warm-blooded species, and/or sex, age, weight and individual condition of the warm-blooded animal.

Normally the dosage is such that a single dose of zoledronic acid or salt or hydrate thereof from 0.002-20.0 mg/kg, especially 0.01-10.0 mg/kg, is administered to a warm-blooded animal weighing approximately 75 kg. If desired, this dose may also be taken in several, optionally equal, partial doses. Doses of zoledronic acid or salts or hydrates thereof in the range from about 0.5 mg to about 20 mg, preferably from about 1 mg to about 10 mg, more preferably 5 mg, may be used for treatment of human patients.

Where the zoledronic acid or salt or hydrate thereof is given intravenously, the 5 mg dose is generally administered over a 15-minute period although shorter and longer periods are possible.

“mg/kg” means mg drug per kg body weight of the mammal—including man—to be treated.

In accordance with the present invention, the zoledronic acid is dosed at intervals of at least about once every three months, six months, e.g., once every 180 days, or less frequently, conveniently once a year, or at any interval in between, e.g., once every 7, 8, 9, 10 or 11 months. Dosing intervals of greater than once per year may be used, e.g., about once every 18 months or about once every 2 years, or even less frequently, e.g., a frequency of up to about once every 3 years or less often.

The dose mentioned above, either administered as a single dose (which is preferred) or in several partial doses, is preferably administered once per year (understanding, of course, that it may not be exactly one year to date but rather at yearly check-ups).

Formulations in single dose unit form contain preferably from about 1% to about 90%, and formulations not in single dose unit form contain preferably from about 0.1% to about 20%, of the zoledronic acid active ingredient. Single dose unit forms, such as capsules, tablets or drages contain, e.g., from about 1 mg to about 500 mg of the zoledronic acid active ingredient.

Pharmaceutical preparations for enteral and parenteral administration are, for example, those in dosage unit forms, such as drages, tablets or capsules and also ampoules. They are prepared in a manner known per se, for example, by means of conventional mixing, granulating, confectioning, dissolving or lyophilizing processes.

For example, pharmaceutical preparations for oral administration can be obtained by combining the active ingredient with solid carriers, where appropriate granulating a resulting mixture, and processing the mixture or granulate, if desired or necessary after the addition of suitable adjuncts, into tablets or drage cores. Suitable carriers are especially fillers, such as sugars, for example, lactose, saccharose, mannitol or sorbitol, cellulose preparations and/or calcium phosphates, for example, tricalcium phosphate or calcium hydrogen phosphate, and also binders, such as starch pastes, using, for example, corn, wheat, rice or potato starch, gelatin, tragacanth, methylcellulose and/or polyvinylpyrrolidone and, if desired, disintegrators, such as the above-mentioned starches, also carboxymethyl starch, cross-linked polyvinylpyrrolidone, agar or alginic acid or a salt thereof, such as sodium alginate. Adjuncts are especially flow-regulating agents and lubricants, for example, silicic acid, talc, stearic acid or salts thereof, such as magnesium or calcium stearate, and/or polyethylene glycol. Drage cores are provided with suitable coatings that may be resistant to gastric juices, there being used, inter alia, concentrated sugar solutions that optionally contain gum arabic, talc, polyvinylpyrrolidone, polyethylene glycol and/or titanium dioxide, or lacquer solutions in suitable organic solvents or solvent mixtures or, to produce coatings that are resistant to gastric juices, solutions of suitable cellulose preparations, such as acetylcellulose phthalate or hydroxypropylmethylcellulose phthalate. Coloring substances or pigments may be added to the tablets or drage coatings, for example for the purpose of identification or to indicate different doses of active ingredient.

Other orally administrable pharmaceutical preparations are thy-filled capsules made of gelatin, and also soft, sealed capsules made of gelatin and a plasticizer, such as glycerol or sorbitol. The dry-filled capsules may contain the active ingredient in the form of a granulate, for example, in admixture with fillers, such as lactose; binders, such as starches; and/or glidants, such as talc or magnesium stearate, and, where appropriate, stabilizers. In soft capsules, the active ingredient is preferably dissolved or suspended in suitable liquids, such as fatty oils, paraffin oil or liquid polyethylene glycols, it being possible also for stabilizers to be added.

Parenteral formulations are especially injectable fluids that are effective in various manners, such as intra-arterially, intramuscularly, intraperitoneally, intranasally, intradermally, subcutaneously or preferably intravenously. Such fluids are preferably isotonic aqueous solutions or suspensions which can be prepared before use, for example, from lyophilized preparations which contain the active ingredient alone or together with a pharmaceutically acceptable carrier. The pharmaceutical preparations may be sterilized and/or contain adjuncts, for example preservatives, stabilizers, wetting agents and/or emulsifiers, solubilizers, salts for regulating the osmotic pressure and/or buffers.

Suitable formulations for transdermal application include an effective amount of the zoledronic acid active ingredient with carrier. Advantageous carriers include absorbable pharmacologically acceptable solvents to assist passage through the skin of the host. Characteristically, transdermal devices are in the form of a bandage comprising a backing member, a reservoir containing the compound optionally with carriers, optionally a rate controlling barrier to deliver the active ingredient of the skin of the host at a controlled and predetermined rate over a prolonged period of time, and means to secure the device to the skin.

In another embodiment, the present invention relates to a formulation that includes zoledronic acid, a form of Vitamin D and optionally calcium in an essentially homogeneous mixture, wherein a solution or solid unit dose can be administered in a single dose.

In some applications, it may be advantageous to utilize the active agent in a “vectorized” form, such as by encapsulation of the active agent in a liposome or other encapsulant medium, or by fixation of the active agent, e.g., by covalent bonding, chelation, or associative coordination, on a suitable biomolecule, such as those selected from proteins, lipoproteins, glycoproteins, and polysaccharides.

### EXAMPLES

The following representative kits provide guidance for appropriate dosages and timeframe for administration:

Kit #1 comprises 7 to 21 tablets of cholecalciferol in unit doses of about 1000 International units for administration for 7 to 21 consecutive days. The kit further includes a sufficient amount of calcium carbonate tablet having a dosage of from about 250 to 1000 mg of calcium carbonate to be administered twice a day during the dosing with the Vitamin D thereby normalizing vitamin D levels and providing adequate amounts of calcium. The kit also include a single dose of from about 3 mg to 7 mg of zoledronic acid which can be administered intravenously over 15 minutes once the two weeks of calcium and vitamin D therapy has been completed.

Kit #2 comprises three (3) capsules of ergocalciferol, in a dose of 50,000 International Units per capsule. Additionally there are eighteen (18) tablets of 500 mg calcium carbonate tables, and a vial having 5 mg of zoledronic acid 5 mg which can be administered over 15 minutes once the ergocalciferol and calcium carbonate have been administered. The ergocalciferol capsules will be administered every other or every third day for six or nine consecutive days along with 500 mg of calcium carbonate administered twice a day. This dosing regime provide a more rapid correction of the vitamin D levels and allow the intravenous bisphosphonate to be administered within seven to 10 days of beginning the calcium therapy.

Kit #3 comprises a single dose of an intramuscular form of cholecalciferol, including 100,000 units/cc or 100,000 units/0.5 cc which will be administered as an intramuscular dose. The kits additionally includes six (6) tablets of calcium carbonate 500 mg and a vial having 5 mg of zoledronic acid which can be administered over 15 minutes three days after the patient has received the intramuscular cholecalciferol and calcium. This kit is useful for patients who are severely vitamin D deficient and require bisphosphonate therapy as soon as possible.

Kit #4 comprises 28 tablets of 0.25 mcg of the vitamin D metabolite, calcitriol, for administration twice a day for two weeks. Additionally the kit includes twenty-eight (28) tablets of 500 mg calcium carbonate and a vial of 5 mg of zoledronic acid 5 mg which is administered intravenously over 15 minutes and two weeks after the patient has received the calcitriol and calcium. This kit is useful in patients who have renal impairment of creatinine clearances between 60 and 30 cc/minutes. Notably, the calcitriol reduces fall rates in patients with reduced creatinine clearances.

Kit #5 comprises twenty-eight (28) tablets of the oral vitamin D metabolite, calcidiol, 1000 International Units for administration of twice daily for two weeks. The kit further includes twenty-eight (28) tablets of 500 mg of calcium carbonate and a vial of 5 mg of zoledronic acid to be administered intravenously over 15 minutes immediately after completion of the two weeks of administration of calcidiol and calcium. This vitamin D metabolite is useful in patients with chronic liver disease who have reduced synthesis of calcidiol; patients contemplating an orthotopic liver transplantion and/or need treatment for their osteoporosis.

Kit #6 comprises the oral vitamin D analogue, doxercalciferol, in a dose unit of 1 microgram for administration three times a week during dialysis sessions or three times a week for patients with renal failure not yet on dialysis. The kit additionally includes twenty-eight (28) 500 mg calcium carbonate tablets and a vial of zoledronic acid which is administered intravenously over 15 minutes. The timing of the zoledronic acid administration is two to four weeks after starting doxercalciferol. This vitamin D analogue is useful in suppressing parathyroid hormone levels and controlling the hypercalcemia seen with calcium supplementation in patients with chronic renal failure. Again the high fracture rate in this group of patients necessitates strategies for treating vitamin D deficiency and still giving the patient an intravenous bisphosphonate.

Kit #7 comprises 14 tablets of cholecalciferol in unit doses of 2000 International units for administration for 14 consecutive days. The kit further includes 28 tablets of calcium carbonate having a dosage of from about 500 mg to be administered twice a day during the dosing with the Vitamin D thereby normalizing vitamin D levels and providing adequate amounts of calcium. The kit also include a single dose of about 5 mg of zoledronic acid which can be administered intravenously over 15 minutes once the two weeks of calcium and vitamin D therapy has been completed.

Kit #8 comprises 7 to 36 tablets of cholecalciferol in unit doses of about 1000 to 3000 International units for administration for 7 to 36 consecutive days. The kit further includes a sufficient amount of calcium carbonate tablet having a dosage of from about 250 to 1000 mg of calcium carbonate to be administered twice a day during the dosing with the Vitamin D thereby normalizing vitamin D levels and providing adequate amounts of calcium. The kit also include a single dose of from about 3 mg to 7 mg of zoledronic acid which can be administered intravenously over 15 minutes once the two weeks of calcium and vitamin D therapy has been completed.

Kit #9 comprises fourteen (14) capsules of cholecalciferol in a dose of 400 International Units per capsule for 14 days of consecutive days. The kit further includes a sufficient amount of calcium carbonate tablet having a dosage of from about 250 to 1000 mg of calcium carbonate to be administered twice a day during the dosing with the Vitamin D thereby normalizing vitamin D levels and providing adequate amounts of calcium. The kit also include a single dose of from about 3 mg to 7 mg of zoledronic acid which can be administered intravenously over 15 minutes once the dosing of calcium and vitamin D therapy has been completed.

### Example 1

Preparation of Capsules Containing Coated Pellets of Active Ingredient

| | |
|---|---|
| Core pellet: | |
| Active ingredient (ground) | 197.3 mg |
| Microcrystalline cellulose | 52.7 mg |
| (Avicel ® PH 105) | |
| | 250.0 mg |
| +Inner coating: | |
| Cellulose HP-M 603 | 10.0 mg |
| Polyethylene glycol | 2.0 mg |
| Talc | 8.0 mg |
| | 270.0 mg |
| +Gastric juice-resistant outer coating: | |
| Eudragit ® L 30 D (solid) | 90.0 mg |
| Triethyl citrate | 21.0 mg |
| Antifoam ® AF | 2.0 mg |
| Water | |
| Talc | 7.0 mg |
| | 390.0 mg |

A mixture of active ingredient with Avicel® PH 105 is moistened with water and kneaded, extruded and formed into spheres. The dried pellets are then successively coated in the fluidized bed with an inner coating, consisting of cellulose HP-M 603, polyethylene glycol (PEG) 8000 and talc, and the aqueous gastric juice-resistant coat, consisting of Eudragit L 30 D, triethyl citrate and Antifoam®. The coated pellets are powdered with talc and filled into capsules (capsule size 0) by means of a commercial capsule filling machine, for example, Hofliger and Karg.

### Example 2

Monolith Adhesive Transdermal System Containina 1-hydroxy-2-(imidazol-1-yl-)-ethane-1,1-diphosphonic acid (zoledronic acid)

Composition:

| | |
|---|---|
| Polyisobutylene (PIB) 300 | 5.0 g |
| (Oppanol B1, BASF) | |
| PIB 35000 | 3.0 g |
| (Oppanol B10, BASF) | |
| PIB 1200000 | 9.0 g |
| (Oppanol B100, BASF) | |
| Hydrogenated hydrocarbon resin | 43.0 g |
| (Escorez 5320, Exxon) | |
| 1-dodecylazacycloheptan-2-one | 20.0 g |
| (Azone, Nelson Res., Irvine/CA) | |
| Active Ingredient | 20.0 g |
| Total | 100.0 g |

### Preparation:

The above components are together dissolved in 150 g of special boiling point petroleum fraction 100-125 by rolling on a roller gear bed. The solution is applied to a polyester film by means of a spreading device using a 300 mm doctor blade, giving a coating of about 75 g/m² After drying (15 minutes at 60° C.), a silicone-treated polyester film (thickness 75 mm, Laufenberg) is applied as the peel-off film. The finished systems are punched out in sizes in the wanted form of from 5-30 cm² using a punching tool. The complete systems are sealed individually in sachets of aluminized paper.

### Example 3

Vial Containing 1.0 mg Dry, Lyophilized 1-hydroxy-2-(imidazol-1-yl)ethane—1,1-diphosphonic acid (mixed sodium salts thereof)

After dilution with 1 mL of water, a solution (concentration 1 mg/mL) for i.v. infusion is obtained.

### Composition:

| | | |
|---|---|---|
| Active Ingredient (free diphosphonic acid) | 1.0 | mg |
| Mannitol | 46.0 | mg |
| Trisodium Citrate × 2 H₂O ca. | 3.0 | mg |
| Water | 1 | mL |
| Water for Injection | 1 | mL |

In 1 mL of water, the active ingredient is titrated with trisodium citrate X 2H₂O to pH 6.0. Then, the mannitol is added and the solution is lyophilized and the lyophilisate filled into a vial.

### Example 4

Ampoule Containing Active Ingredient Dissolved in Water. The solution (concentration 3 mg/mL) is for i.v. infusion after dilution.

### Composition:

| | | |
|---|---|---|
| Active ingredient | 19.73 | mg |
| (Δ 5.0 mg of anhydrous active ingredient) | | |
| Mannitol | 250 | mg |
| Water for injection | 5 | mL |

### Example 5

Treatment of Patients

“A Multinational, multicenter, double-blind, randomized, placebo-controlled, parallel-group study assessing the efficacy of intravenous zoledronic acid in preventing subsequent osteoporotic fractures after a hip fracture” is undertaken.

### Study Objectives

The primary objective is to demonstrate, that a kit comprising a single dose of zoledronic acid, (5 mg i.v. over 15 minutes) plus precursor dosing regime for a number of continuous or intermittent days of 50,000-125,000 units of vitamin and elemental calcium (1000-1500 mg p.o. daily in a divided dose) before the dose of zoledronic acid to men and women after surgical repair of low-trauma hip fracture will significantly reduce the rate of clinical fractures, defined as all subsequent osteoporotic fractures, compared to a dosing regime that dose not include the zoledronic acid.

Secondary Objective is to:
Demonstrate an increase in Bone Mineral Density (BAD) in the total hip and femoral neck, using dual X-ray absorptiometry (DXA), of the non-fracture hip at:
   12 months compared to randomization (Baseline)
   24 months compared to randomization (Baseline)
   After 24 months, an annual BMD will be compared to randomization (Baseline)

Total hip BMD and femoral neck BMD will be collected by DXA at each investigator site and reported on CRF page at randomization visit and every 12 month visit thereafter. Percentage change from baseline of total hip BMD and femoral neck BMD will be computed for each of those patients, and used for the analysis.

### Overall Study Design

This will be a multicenter, randomized, double-blind, placebo-controlled, parallel group trial in men and women. Patients undergoing recent surgical repair of a low-trauma hip fracture will be eligible for enrollment. There will be at least 3 study contacts with patients after randomization within the first 24 months. Patients will have additional visits annually until 211 patients have reached the primary endpoint. The final visit for patients will be contingent on when this endpoint is achieved. Once the endpoint is achieved all patients will need to come in for a final visit. The final visit will be no less than 30 days from the patient's last dose of study medication or no more than 90 days after the day 211 patients have reached the primary endpoint.

All patients signing informed consent at screening will receive a kit comprising a 14 day supply of a form of Vitamin D and calcium containing tablets to be ingested before administration of the zoledronic acid or a placebo. The kit may further include a single loading dose of a form of Vitamin D in a high dosing concentration before the administration of the 14 day supply of a lower dose of Vitamin D.

Subjects will be contacted every 3 months between annual visits. Self-reports of fractures, will be recorded at each study contact and at any time identified during the study.

Hip BMD will be measured using dual X-ray absorptiometry (DXA) of the non-fracture hip at randomization and every 12 months during the study. If the site is unable to measure hip BMD of non-fracture hip then a lumbar spine BMD will be measured for safety only.

Concomitant medications and co-morbid illnesses will be recorded at each study visit and vital signs will be measured at randomization. Height and weight will be measured at, randomization, 12 months and every annual visit, thereafter. A chemistry panel will be measured at randomization, 12 months and every 12 months, thereafter. Calculated creatinine clearance will also be determined within 4 weeks of randomization, 12 month visit and every 12 months, thereafter. The calculated creatinine clearance must be known within 4 weeks prior to giving study drug to the patient. A CBC will be performed at visit 1 for baseline safety assessment of eligibility.

Chest and hip X-rays will be collected at screening. Hip X-ray of the incident hip will be performed at 6 months. Hip fracture healing will be assessed using clinical signs and symptom (persistent pain and/or inability to bear weight on index hip) at randomization, 6 and 12 months following the index fracture repair. All subjects reporting these signs or symptoms will have hip radiographs reviewed by the CEC.

### Patient Population

The study population will consist of men and women aged 50 years and older who have suffered a recent low-trauma, acute hip fracture and were ambulatory prior to the fracture. Patients admitted to orthopedic surgical or medical services, extended care or rehabilitation facilities and seen in clinic will be identified. Eligible patients will be ascertained from hospital admission logs, or operating room logs, extended care facility/rehabilitation logs and clinic schedules. Patients will be randomized to a treatment group, that being receiving a kit that comprises the correct dosage of vitamin D and elemental calcium supplements in addition to either zoledronic acid and or a placebo thereof. Patients will receive the kit that includes instructions for taking the vitamin D and elemental calcium supplements daily and the subsequent administration of the either zoledronic acid and or a placebo thereof.

The investigator or designee must explain to each patient or legally authorized representative the nature of the study, its purpose, the procedures involved, the expected duration, the potential risks and benefits involved and any discomfort it may entail. Each patient must be informed that participation in the study is voluntary, that he/she may withdraw from the study at any time and that withdrawal of consent will not affect subsequent medical treatments or relationships with treating physicians. Informed consent will be given by means of a standard written statement, written in non-technical language. The patient should read and consider the statement before signing and dating it and will be given a copy of the signed document. If written consent is not possible, oral consent can be obtained if witnessed by one or more persons not involved in the study, and the reason why the patient was unable to sign the form must be documented. No patient can enter the study before informed consent has been obtained.

Inclusion Criteria
1. Male or female patient aged greater than or equal to 50 years.
2. Patient may be randomized up to 90 days post-surgical repair of a low-trauma hip fracture.
3. Patient was ambulatory with or without assistive device prior to the hip fracture.
4. Patient must have intact both lower appendages (legs), not an amputee.
5. Patient preferably can comprehend and have the physical mobility to opening the contents of the provided kit and reading the instructions for sequential administration of the components within the kit.

Exclusion Criteria
1. Treatment with any investigational drug within the past 30 days prior to randomization
2. Previous history of allergic reaction or hypersensitivity to bisphosphonates
3. History of uveitis or iritis, except when secondary to trauma, and this must have resolved greater than 2 years prior to randomization.
4. Calculated Creatinine Clearance less than or equal to 30.0 ml/min
5. Serum calcium greater than 2.75 mmoL/L (11.0 mg/dL)
6. Serum alkaline phosphatase, greater than 2.5×ULN
7. Hypocalcemia (serum corrected calcium less than 8 mg/dl or 2.0 mmol/L at screening and/or randomization)
8. Primary hyperparathyroidism, hypoparathyroidism, Osteogenesis imperfecta, Paget's disease, or any other metabolic bone disease, except osteoporosis
9. Cancer Exclusion:
   Patients with a new diagnosis or active treatment for any malignancy less than or equal to 12 months prior to randomization.
   Patients with known metastases (or by history)
   Patients with the following may be included: basal cell or squamous cell carcinoma of the skin, colonic polyps with non-invasive malignancy which have been removed, Ductal Carcinoma in-situ (DCIS) that has been surgically removed, and Carcinoma in-situ (CIS) of the uterine cervix that has been surgically removed)
10. Previous major solid organ transplant recipient less than 2 years ago, prior to randomization or on a transplant waiting list
11. Any prior use of i.v. bisphosphonate within the last 2 years
12. Any prior use of PTH and PTH analogs for more than 1 week; if used for less than or equal to 1 week, wash out period for PTH and PTH analogs is 6 months. The reference point for the washout period should be the date of randomization
13. Any prior use of sodium fluoride treatment for osteoporosis
14. Any prior use of strontium (all formulations)
15. Hip fractures unlikely to be due to osteoporosis (traumatic fracture, malignant fracture, osteomyelitic fracture, hardware related fracture)
16. Serious disease and/or any clinically significant laboratory findings that in the opinion of the investigator could seriously affect the patient's ability to participate in the study or that may limit life expectancy to less than 6 months
17. Other conditions/circumstances likely to lead to poor treatment adherence
18. Pregnant, lactating, or has the potential to become pregnant and does not agree to use an effective contraceptive method

Interruption or Discontinuation of Treatment

Any use of bisphosphonates (other than study therapy), PTH and PTH analogs, fluoride, strontium, or anabolic steroids, except testosterone in the case of hormone replacement therapy in hypogonadal men, for more than two weeks during the study will be the cause for discontinuation of the patient from study treatment. However, all patients will remain in the trial for observation regardless of adherence to study medication.

Patients who have been unblinded will also be discontinued from study treatment.

It will be documented whether or not each patient completed the clinical study. If study treatment or observations are discontinued for a patient, the reason will be recorded.

Reasons that a patient may discontinue participation in a clinical study are considered to constitute one of the following:
1. adverse event(s)
2. abnormal laboratory value(s)
3. abnormal test procedure result(s)
4. unsatisfactory therapeutic effect
5. subject's condition no longer requires study treatment
6. protocol violation
7. subject withdrew consent
8. lost to follow-up
9. administrative problems
10. death

The IVRS must also be called and the patient's discontinuation reported accordingly.

Follow-up for patients no longer on study medications will include fracture information, serious and non-serious AEs and all other regular study measurements (labs, DXA, X-rays). These patients will continue to be provided with calcium and vitamin D supplements. For patients who do not wish to continue with follow-up visits or withdraw consent, the final visit CRFs and procedures will be completed. However, the DXA and X-rays should not be performed if performed in the preceding 3 months, unless there is clinical cause to do so.

Patient specific double blinded drug kits will be prepared by Clinical Trial Services (CTS). Active drug kits will contain vials of zoledronic acid (5 mg in 5 ml of sterile water for injection) and 2 (10 mL) physiologic (0.9%) normal saline for the flushing of the intravenous line. The kits will further a sufficient amount of a form of Vitamin D and calcium in an amount sufficient to overcome any Vitamin D deficiency before dosage of zoledronic acid or the placebo.

Patients should be encouraged to have sufficient food and liquid intake, at dosing and for several days following the dose as no special dietary restrictions apply.

Concomitant Medications:

Due to the age of the patient population studied, it is likely that many patients will have other significant medical problems requiring medications as therapy. Since many common medications affect bone metabolism, an accurate account of these medications must be documented during the period of the study. Patients will be asked about use of the medications below. The name of the drug should be recorded on the concomitant medication/therapy page in the CRF.

The following medications will not be allowed during the study:
a) Bisphosphonates other than the study medication
b) Sodium fluoride
c) PTH and PTH analogs
d) Anabolic steroids except testosterone in the case of hormone replacement therapy in hypogonadal men
e) Strontium
f) Any investigational therapy other than the study medication

At each patient contact, patients will be queried on the use of concomitant medications. Since this trial will compare usual medical care for patients with hip fractures against zoledronic acid, patients will be allowed to receive all approved therapies for osteoporosis except those mentioned above. Since less than 10% of hip fracture patients receive any therapy for their osteoporosis, the use of concomitant osteoporosis therapies (calcitonin, SERMs (e.g. raloxifene), hormone replacement therapy (HRT), tibolone, DHEA(s), ipriflavone, and testosterone, as hormone replacement in the case of hypogonadal men), with the exception of those listed above, is not anticipated to affect the outcome of this trial.

Primary Endpoint

Clinically evident fractures occurring in the follow-up period are the primary endpoint. Facial, skull and digital fractures are not associated with osteoporosis and will not qualify as an outcome. The site study coordinator will obtain copies of radiology reports or physician's chart documentation of X-ray results for non-vertebral fractures. These will be submitted to the Clinical Endpoint Committee (CEC) within 1 week of the patient's report of the event. Radiographs will also be requested for some fractures that require further confirmation.

The diagnosis of clinically evident vertebral fracture will require the following: 1) acute onset or worsening back pain in a localized area of the spine as triggered in the CRF, and 2) PA and lateral lumbar spine films (obtained during routine clinical care) showing one or more grade of vertebral height loss by the semi-quantitative technique of Genant et al in comparison with baseline X-rays. For men, a modification of the Genant criteria will be used in order to improve specificity for vertebral fracture. If no baseline films are available, an incident fracture will be defined as one or more vertebrae with grade 2 or higher deformity. If modalities other than plain film are used to diagnose the vertebral fracture, an incident fracture is defined as a significant deformity in a vertebrae with no greater than grade 1 deformity at baseline.

If a patient suffers more than one fracture after hip fracture, the first such event will be counted as the primary endpoint. If a patient suffers more than one fracture at the same time, the most clinically serious will be counted as the primary endpoint in the following hierarchy: hip, long bone, vertebral, wrist, other.

Both traumatic and minimally traumatic fractures will be considered endpoints. New fractures associated with orthopedic hardware are of importance and will also be considered primary endpoints. Fractures judged by the CEC to be due to metastatic cancer, osteomyelitis or high energy trauma (eg. motor vehicle collision or falls from greater than standing height) will not be considered endpoints.

Secondary Endpoints
1. Demonstrate an increase in Bone Mineral Density (BMD) of the total hip and femoral neck BMD, using dual X-ray absorptiometry (DXA) of the non-fracture hip at:
2. 12 months compared to randomization (Baseline)
3. 24 months compared to randomization (Baseline)
4. After 24 months, an annual BMD will be compared to randomization (Baseline)

Total hip BMD and femoral neck BMD will be measured by DXA at each investigator site and reported on CRF page at randomization visit and every 12 month visit thereafter. Percentage change from baseline of total hip BMD and femoral neck BMD will be computed for each of those patients, and used for the analysis.

Statistical Methods

The study is designed to show superiority of the kit containing a dosing supply of a form of Vitamin D and a calcium containing component in combination with zoledronic acid and instructions for correct chronological administration of same. zoledronic acid compared to placebo in reducing clinical fracture rate after surgical repair of low-trauma hip fracture. The primary endpoint is “time to event” (time from randomization to first clinical fracture), and log-rank test will be used in the primary analysis.

Efficacy Evaluation

The analysis of primary efficacy variable will be performed on the intent-to-treat and per-protocol populations. The primary analysis population is intent-to-treat population. The analysis of secondary efficacy variables will be performed on intent-to-treat population. In addition, adjustment for multiple comparisons will not be made for any of the secondary efficacy variables

Primary Efficacy Analysis

The primary efficacy endpoint is time to first clinical fracture. Between treatment differences will be evaluated using a log-rank test to compare the time to clinical fracture between the two treatment groups. The Kaplan-Meier estimates of the time to clinical fracture for each treatment will be plotted. The Kaplan-Meier estimates of incidence of clinical fractures at the end of study will be presented.

The calculation of time to clinical fracture event will be determined based on the following methods:

1. If a clinical fracture has occurred, the time to clinical fracture event will be computed from the date of receiving the kit and initiating administration of the Vitamin D to the detection date of fracture.

2. If a clinical fracture has not occurred and the subject completes the study, the censoring time will be computed from the date of receiving the kit and initiating administration of the Vitamin D to the last study visit.

3. If a subject dies without a clinical fracture, then the censoring time will be computed from the date of receiving the kit and initiating administration of the Vitamin D to the date of death.

4. If a subject is lost to follow-up without a clinical fracture, then the censoring time will be computed from the date of receiving the kit and initiating administration of the Vitamin D to the last available visit date in the final database.

Since the clinical fracture rate in some centers/countries can be very small or even zero, centers/countries will be pooled into regions to assess any geographical differences that may exist between treatments. A pooling scheme will be decided before unblinding. Tabular and/or graphical methods will be used to assess treatment-by-region interaction as appropriate (secondary analysis on primary endpoint)

Since one subject can have more than one clinical fracture (multiple events data), as an exploratory analysis, the Anderson-Gill type approach will be used to explore the between-treatment differences with respect to all clinical fracture rates.

Secondary Efficacy Analysis

The secondary efficacy endpoints are the percent change relative to randomization of total hip BMD and femoral neck BMD at month 12, 24 and every 12 months thereafter after receiving the kit and initiating Vitamin D and optionally calcium treatment.

Percent change from randomization at each visit will be analyzed for BMD efficacy variables. Between-treatment differences will be evaluated using a two-way analysis of variance (ANOVA) model with treatment and center as explanatory variables.

A positive outcome is indicated by both the 12-month and 24-month results showing a significant reduction in secondary osteoporotic fractures in patients who have recently undergone surgical repair of a hip fracture when they received the kit and initiated consumption of the Vitamin D before the dose of zoledronic acid vs. those patients that received a kit that include a placebo for zoledronic acid.

That which is claimed is:

1. A kit for reducing secondary osteoporotic skeletal fractures in subject at risk for vitamin D deficiency, the kit comprising a therapeutically effective amount of a bisphosphonate or analogue thereof in combination with an effective amount of Vitamin D, a metabolite thereof, a precursor thereof or an analogue thereof, wherein the Vitamin D is packaged in daily doses for administration before the bisphosphonate.

2. The kit of claim 1, wherein the Vitamin D is in the form of cholecalciferol, calcidiol, ergocalciferol, dihydrotachysterol, doxercalciferol, paricalcitol or calcitriol.

3. The kit of claim 1, wherein the bisphosphonate is zoledronic acid.

4. The kit of claim 1, further comprising calcium.

5. A multi-component kit for a patient at risk for secondary osteoporotic skeletal fractures and vitamin D deficiency, the kit comprising (a) Vitamin D or functional analogue thereof; (b) zoledronic acid or functional analogue thereof and optionally a calcium containing component.

6. A method for protecting a patient at risk for secondary osteoporotic skeletal fractures and vitamin D deficiency comprising the steps of:
providing a container sized for inclusion of a sufficient amount of Vitamin D for a dosing regime comprising multiple dosages and a single dosage of a bisphosphonate; and
providing instructions for the dosing regime.

7. The method of claim 6, wherein the container further comprising multiple doses of calcium.

8. The method of claim 6, wherein the bisphosphonate is zoledronic acid.

9. The method of claim 6, wherein the container is configured to be permissive for the removal of a single dosage of Vitamin D on a daily basis and removal of same is evidenced by an empty dosage spot in the container.

10. The method of claim 8, wherein the container is configured to include a tablet, transdermal patch, syringe or vial of a single dosage of zoledronic acid or analogue thereof.

11. A composition kit comprising at least two portions wherein multiple doses of Vitamin D or an analogue thereof are packaged in a first portion and at least one dose of zoledronic acid is packaged in a second portion

12. A kit comprising at least a first and a second product, wherein the second product is administered to a subject after sequentially administering the first product, and wherein the first product comprises a form of Vitamin D and the second product is zoledronic acid, a salt or hydrate thereof.

13. The kit of claim 12, further comprising a third product of a calcium containing compound.

14. A method of treating bone disease and Vitamin D deficiency, comprising:
a) opening a sealed disposable package, wherein the package comprises multiple single-unit doses of Vitamin D and optionally calcium;
b) administering a single-unit dose, wherein steps a) to b) are repeated daily for a period of 10 to 31 days; and
c) opening a vial contained within the sealed disposable package, wherein the vial comprises a single-unit dose of zoledronic acid or analogue thereof.

15. The method of claim 14, wherein the steps a) to c) are conducted from 10 to 120 days after a bone fracture.
